(19) United States
(12) Patent Application Publication
(10) Pub. No.: US 2006/0015023 A1
(43) Pub. Date: Jan. 19, 2006

(54) Preparation kit for noninvasive glucose concentration determination

(76) Inventors: Stephen L. Monfre, Gilbert, AZ (US); Kevin H. Hazen, Gilbert, AZ (US)

Correspondence Address: GLENN PATENT GROUP, 3475 EDISON WAY, SUITE L, MENLO PARK, CA, 94025, US

(21) Appl. No.: 10/866,373

(22) Filed: Jun. 10, 2004

(51) Int. Cl. A61B 5/00; B65D 69/00; A61L 2/00

(52) U.S. Cl. 600/344; 422/294; 206/229

(57) ABSTRACT

The invention involves a kit for use in conjunction with a near-infrared, noninvasive glucose concentration analyzer. The preferred kit contains a set of elements used in preparation of a sample site and/or for use in a sampling process. The invention comprises a kit that includes any of a guide, a plug, an adhesive, an alignment tool, a cleaner, contact fluid, and a package for use in combination with a noninvasive glucose concentration analyzer.

### CROSS REFERENCE TO RELATED APPLICATIONS

This document claims priority to U.S. patent application Ser. No. 10/170,921, filed Jun. 12, 2002 (attorney docket no. IMET0045CIP), which claims priority to U.S. patent application Ser. No. 09/563,782 filed May 2, 2000 now U.S. Pat. No. 6,415,167, and to U.S. patent application Ser. No. 10/472,856, filed Sep. 18, 2003 (attorney docket no. SENS0011), which claims priority to PCT application no. PCT/US03/07065, filed Mar. 7, 2003, which claims priority to U.S. provisional patent application No. 60/362,885, filed Mar. 8, 2002, all of which are incorporated herein in their entirety by this reference thereto.

### BACKGROUND OF THE INVENTION

1. Technical Field

The invention relates generally to biomedical methods and apparatus. More particularly, the invention relates to a kit for preparing a tissue sample site for noninvasive glucose concentration analysis.

2. Description of Related Art

Diabetes

Diabetes is a chronic disease that results in improper production and use of insulin, a hormone that facilitates glucose uptake into cells. While a precise cause of diabetes is unknown, genetic factors, environmental factors, and obesity appear to play roles. Diabetics have increased risk in three broad categories: cardiovascular heart disease, retinopathy, and neuropathy. Complications of diabetes include: heart disease and stroke, high blood pressure, kidney disease, neuropathy (nerve disease and amputations), retinopathy, diabetic ketoacidosis, skin conditions, gum disease, impotence, and fetal complications. Diabetes is a leading cause of death and disability worldwide. Moreover, diabetes is merely one among a group of disorders of glucose metabolism that also includes impaired glucose tolerance, and hyperinsulinemia, or hypoglycemia.

Noninvasive Glucose Determination

Noninvasive analyses allow for rapid and painless estimation of analyte concentrations, such as glucose. The rapid and painless nature of the test facilitates additional and more frequent glucose concentration determination, which is associated with an increased ability to manage diabetes.

There exist a number of noninvasive approaches for glucose concentration determination. These approaches vary widely, but have at least two common steps. First, an apparatus is used to acquire a reading from the body without obtaining a biological sample. Second, an algorithm is used to convert this reading into a glucose concentration determination.

One species of noninvasive glucose concentration analyzer are those based upon spectra. Typically, a noninvasive apparatus uses some form of spectroscopy to acquire the signal or spectrum from the body. Noninvasive spectroscopic techniques include Raman and fluorescence as well as techniques using light from ultraviolet through the infrared [ultraviolet (200 to 400 nm), visible (400 to 700 nm), near-IR (700 to 2500 nm or 14,286 to 4000 cm⁻¹), and infrared (2500 to 14,285 nm or 4000 to 700 cm⁻¹)]. A particular range for noninvasive glucose concentration determination in diffuse reflectance mode is about 1100 to 2500 nm or ranges therein. See K. Hazen, Glucose Determination in Biological Matrices Using Near-Infrared Spectroscopy, doctoral dissertation, University of Iowa, 1995. Alternative ranges include 1100 to 1900 nm and ranges therein. Noninvasive techniques are used on the surface of the body and with sample sites including: a hand, finger, palmar region, base of thumb, back of wrist, forearm, volar aspect of the forearm, dorsal aspect of the forearm, upper arm, head, earlobe, eye, tongue, chest, torso, abdominal region, thigh, calf, foot, plantar region, and toe.

Instrumentation

Noninvasive glucose concentration estimation using a near-infrared analyzer generally involves the illumination of a region of the body with light, such as with near-infrared electromagnetic radiation. The light is partially absorbed and partially scattered according to its interaction with the constituents of the tissue prior to exiting the sample and being directed to a detector. The detected light contains quantitative information that corresponds to the known interaction of the incident light with components of the body tissue including water, fat, protein, and glucose.

Chemometric calibration techniques extract the glucose related signal from the measured spectrum through various methods of signal processing and calibration, including one or more mathematical models. The models are developed through the process of calibration on the basis of an exemplary set of spectral measurements known as the calibration set and associated set of reference blood glucose concentrations based upon an analysis of fingertip capillary blood or venous blood. Common analyses include an nth derivative and multivariate regression.

Dynamic Properties of Skin

The dynamic properties of skin tissue is an important aspect of noninvasive glucose concentration determination. At a given measurement site, skin tissue is often assumed to remain static, except for changes in the target analyte concentration and the concentration of other interfering species. However, variations in the physiological state and fluid distribution of tissue profoundly affect the optical properties of tissue layers and compartments over a relatively short period of time.

Many factors impact the physical and chemical state of skin. These include environmental and physiological factors. These factors include at least body temperature and environmental temperature. Often control of these variables is critical to analyte estimation precision and accuracy when multivariate techniques are used in analyses.

### PRIOR ART

There exist a number of reports on noninvasive glucose concentration determination technologies. Some of these relate to general instrumentation configurations required for noninvasive glucose concentration determination while others refer to sampling technologies. Those that provide an overview of the noninvasive glucose concentration determination field are briefly reviewed here.

Need

There are a number of studies documenting the need for an accurate and precise noninvasive glucose concentration analyzer, such as R. Barnes, J. Brasch, D. Purdy, W. Lougheed, Non-invasive determination of analyte concentration in body of mammals, U.S. Pat. No. 5,379,764 (Jan. 10, 1995) that describe a noninvasive glucose concentration determination analyzer that uses data pretreatment in conjunction with a multivariate analysis to determine blood glucose concentrations.

General Instrumentation

P. Rolfe, Investigating substances in a patient's bloodstream, UK patent application no. 2,033,575 (Aug. 24, 1979) describe an apparatus for directing light into the body, detecting attenuated backscattered light, and using the collected signal to determine glucose concentrations in or near the bloodstream.

C. Dahne, D. Gross, Spectrophotometric method and apparatus for the non-invasive, U.S. Pat. No. 4,655,225 (Apr. 7, 1987) describe a method and apparatus for directing light into a patient's body, collecting transmitted or backscattered light, and determining glucose concentration from selected near-IR wavelength bands. Wavelengths include 1560 to 1590, 1750 to 1780, 2085 to 2115, and 2255 to 2285 nm with at least one additional reference signal from 1000 to 2700 nm.

M. Robinson, K. Ward, R. Eaton, D. Haaland, Method and apparatus for determining the similarity of a biological analyte from a model constructed from known biological fluids, U.S. Pat. No. 4,975,581 (Dec. 4, 1990) describe a method and apparatus for measuring a concentration of a biological analyte, such as glucose concentration, using infrared spectroscopy in conjunction with a multivariate model. The multivariate model is constructed form plural known biological fluid samples.

J. Hall, T. Cadell, Method and device for measuring concentration levels of blood constituents non-invasively, U.S. Pat. No. 5,361,758 (Nov. 8, 1994) describe a noninvasive device and method for determining analyte concentrations within a living subject using polychromatic light, a wavelength separation device, and an array detector. The apparatus uses a receptor shaped to accept a fingertip with means for blocking extraneous light.

S. Malin, G Khalil, Method and apparatus for multi-spectral analysis of organic blood analytes in noninvasive infrared spectroscopy, U.S. Pat. No. 6,040,578 (Mar. 21, 2000) describe a method and apparatus for determination of an organic blood analyte using multi-spectral analysis in the near-infrared (NIR). A plurality distinct nonoverlapping regions of wavelengths are incident upon a sample surface, diffusely reflected radiation is collected, and the analyte concentration is determined via chemometric techniques.

Temperature

K. Hazen, Glucose Determination in Biological Matrices Using Near-Infrared Spectroscopy, doctoral dissertation, University of Iowa (1995) describe the adverse effect of temperature on near-IR based glucose concentration determinations. Physiological constituents have near-IR absorbance spectra that are sensitive, in terms of magnitude and location, to localized temperature and the sensitivity impacts noninvasive glucose concentration determination.

Guide

T. Blank, G. Acosta, M. Mattu, S. Monfre, Fiber optic probe guide placement guide, U.S. Pat. No. 6,415,167 (Jul. 2, 2002) describe a contact fluid of one or more perfluoro compounds where a quantity of the contact fluid is placed at an interface of the optical probe and measurement site. Perfluoro compounds do not have the toxicity associated with chlorofluorocarbons. Blank also teaches the use of a guide in conjunction with a noninvasive glucose concentration analyzer to increase precision of the location of the sampled site resulting in increased accuracy and precision in a noninvasive glucose concentration determination. The guide is used for a period of time to increase precision in sampling throughout a period of sampling, such as a fraction of a day, one day, or a period of multiple days.

Coupling Fluid

M. Robinson, Method for noninvasive blood analyte measurement with improved optical interface, U.S. Pat. No. 6,152,876, (Nov. 28, 2000) and M. Rohrscheib, Method and apparatus for non-invasive blood analyte measurement with fluid compartment equilibration, U.S. Pat. No. 6,240,306, (May 29, 2001) describe coupling fluids comprising chlorofluorocarbon compounds and chlorofluorocarbon compounds with additives. No suggestion of contact fluids comprised of fluorocarbons is made.

Equilibration

A number of reports exist describing the difference (or lack of difference) between traditional glucose concentration determinations and alternative site glucose concentration determinations. Some have recognized the potential difference as having an impact upon noninvasive glucose concentration calibration and maintenance.

In-light Solutions (formerly Rio Grande Medical Technologies), has reported the use of heat, rubrifractants, or the application of topical pharmacologic or vasodilating agents, such as nicotinic acid, methyl nicotinamide, minoxidil, nitroglycerin, histamine, menthol, capsaicin, and mixtures thereof to hasten the equilibration of the glucose concentration in the blood vessels with that of the interstitial fluid. See M. Rohrscheib, C. Gardner, M. Robinson, Method and Apparatus for Non-invasive blood analyte measurement with Fluid Compartment Equilibration, U.S. Pat. No. 6,240,306 (May 29, 2001) and M. Robinson, R. Messerschmidt, Method for Non-Invasive Blood Analyte Measurement with Improved Optical Interface, U.S. Pat. No. 6,152,876 (Nov. 28, 2000).

Cleaning Kits

H. Berman, Glucose measurement using non-invasive assessment methods, U.S. Pat. No. 6,522,903, (Feb. 18, 2003) describe a process for cleaning a skin surface prior to mid-infrared attenuated total reflectance (ATR) analysis for noninvasive glucose concentration determination. Cleaning procedures use a glucose solvent, such as water for removing glucose from the sample site, a solvent for removing water, a skin softener, an absorbent, and use of a weak acid. These techniques are also taught in H. Berman, Cleaning system for infrared ATR glucose measurement system (II), U.S. Pat. No. 6,362,144, (Mar. 26, 2002); H. Berman, Method for preparing skin surface and determining glucose levels from that surface, U.S. Pat. No. 6,424,848, (Jul. 23, 2002); H. Berman, Infrared ATR glucose measurement system having an ATR with mirrored ends, U.S. Pat. No. 6,421,548, (Jul. 16, 2002); H. Berman, Method for determining blood glucose levels from a single surface of the skin, U.S. Pat. No. 6,445,938, (Sep. 3, 2002); and H. Berman, Infrared ATR glucose measurement system using a single surface of skin, U.S. Pat. No. 6,430,424, (Aug. 6, 2002). These patents all describe mid-IR techniques based upon ATR, which is a technique that is not comparable to near-IR techniques. In addition, no suggestion of a guide or alignment device is made.

None of these reports discuss a sample preparation kit in relation to noninvasive analyte concentration estimation in the near-infrared.

The body is dynamic in nature. The skin surface is also subject to chemical, physical, and environmental impacts that are known to impact noninvasive glucose concentration determination. It would be advantageous to provide a kit for preparation of a sample site for noninvasive glucose concentration determination that mitigates these issues and provides a contained resource for ease of use.

### SUMMARY OF THE INVENTION

A kit for use in conjunction with a noninvasive analyzer is presented. More particularly, a contained set of elements used in preparation of a sample site and/or for use in a sampling process are presented for use in conjunction with a noninvasive glucose concentration estimation apparatus.

### BRIEF DESCRIPTION OF THE DRAWINGS

FIG. 1 is a perspective view of a guide and plug included in a kit according to the invention;

FIG. 2 is a perspective view of a guide and a photostimulator included in a kit according to the invention; and

FIG. 3 is a perspective view of a photostimulator with an LED and a guide included in a kit according to the invention.

### DETAILED DESCRIPTION OF THE INVENTION

The invention comprises a kit that includes any of a guide, a plug, an adhesive, an alignment tool, a cleaner, contact fluid, and a package for use in combination with a noninvasive glucose concentration analyzer. Each of the guide, plug, adhesive, alignment tool, cleaner, and contact fluid are useful in preparation of a sample site for noninvasive glucose concentration determination. In particular, use of the implements result in increased precision and accuracy in subsequent glucose concentration determinations. Some of these items are required to be clean, are small and readily misplaced, or have an expiration date. Therefore, containment of these items into a single or multiple use kit is desirable in terms of cost, ease of use, cleanliness, and marketing.

Guide

Generally, a guide is a replaceably attached apparatus used as one-half of a lock and key mechanism. For example, a guide is replaceably affixed to a sample site, such as to a subject's skin. Any of various attachments are replaceably attached to the guide or inserted into the guide. A number of guide (lock) configurations exist and a number of attachments (keys) exist. Many of these are previously described in U.S. Pat. No. 6,415,167; U.S. patent application Ser. No. 10/170,921; and provisional application No. 60/472,613, which are all incorporated herein in their entirety by this reference thereto. A guide typically has an attachment side that interfaces to a sample site and an outer side that interfaces to an accessory, such as to the tip of a sampling probe of a sample module that is part of a noninvasive analyzer, a plug, or a photostimulator. For example, input and/or output elements are coupled via the guide to a targeted and controlled sample site.

Lock (Guide)

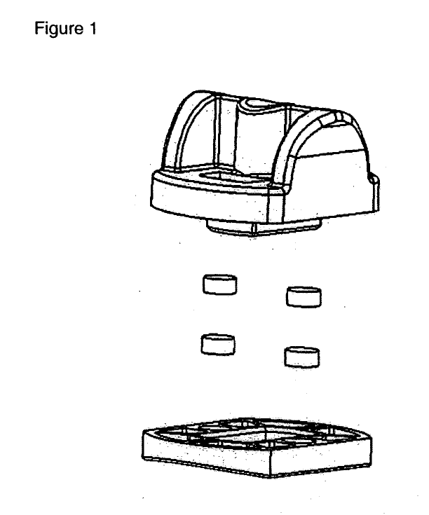

A core feature of the guide element is that is makes up one-half of a lock and key combination. That is, a surface exists that reproducibly guides the other half of a lock and key element into a selected sample position. An example guide 10 is presented in FIG. 1, in relation to a plug 11. Optional magnets 12 are included in this figure and are described below. In this example, the lock element is in the structure of the guide, but alternatively it may be in the attachment. In FIG. 1, the lock element defines an aperture formed hole in the guide that has a roughly rectangular shape with two opposing sides each having rounded shapes. The rectangular shape limits rotational alignment. Preferably, the guide does not have rotational freedom. Rotational freedom is eliminated by flattening one of the round ends. Many lock element shapes are readily used. Examples include virtually any geometrically shaped opening or any shape (not necessarily an opening) that provides reproducible positioning while preferably preventing freedom of rotation. In the particular guide elements presented herein, optional additional holes or divots are pictured. The function of these is primarily to reduce weight, minimize surface abnormalities, such as sink marks on the sampling site, and to maintain strength while limiting the twisting freedom of the guide. An additional optional component, pictured in FIG. 1, is a pair of magnets. The magnets are used to control contact force and/or to aid in alignment of the lock and key mechanism. In the guide pictured, optional opposing pole magnets are also placed into the plug. Of the paired magnets, one half of the pair could be a be metallic substance, such as sheet metal or stainless steel. This is done to reduce cost and/or weight.

Preferably, the contact side of a guide is matched to the shape of the sample because such matching results in increased precision of subsequent optical sampling. For example, an arm sampling site varies between individuals in terms of circumference or radius of curvature. For the case of an arm sampling site, the skinnier the arm the smaller the radius of curvature of the optimal guide. Optional guides include a sample surface with a flat, 5-inch, 4-inch, and 3-inch radius of curvature. At the sample site, the guide surface is preferably flat. Thus, one embodiment of a guide has a surface for interfacing with a sample site, such as a forearm with the shape of the outside sides of a cylinder that is modified to be planar near the sample site. Alternatively, at least a part of the contact surface of the guide is flexible. This allows the weight of an attachment, such as a sample probe, to be distributed over a larger surface area, which reduces the impact of pressure on the sample site.

A guide is attached to a sample site with a number of means, such as a band, a two piece watch-type band, a strap, Velcro, or preferentially with a double sided adhesive. Commonly the adhesive is firmly placed onto the sample site and then the guide is visually aligned onto the adhesive. This sequence reduces the likelihood of the adhesive separating from the sampling site. Optionally, the adhesive is attached to the guide and the pair are placed into contact with the sampling site as a unit. This eases alignment of the guide with the adhesive. The guide and adhesive are semi-permanently and removeably attached to the sample site. The guide is typically left in place for the remainder of a sampling period, such as one waking day or the length of a data collection period, such as two, four, or eight hours.

An optional intermediate layer is used between the guide and the double sided adhesive that attaches to the sampling site. Essentially, this is a semi-flexible material, such as acetate. The material provides some flexibility to allow the sample site skin to stretch. This reduces sampling transients resulting from movement of the subject. Conversely, in subjects with poor turgor, the skin flexes too much and a more rigid insert, such as a plastic film is optionally used.

The guide is preferentially formed out of a thermoplastic, such as a polycarbonate or a polyurethane. However, many other materials may be used, as will be obvious to those skilled in the art. Because the guide is in contact with the sample site (sometimes with an intermediate adhesive), the thermal properties of the guide become important. Typically, the guide is non-thermally conductive to reduce sampling site temperature gradients. However, in some cases a thermally conductive guide is preferred, such as when heat flow to or from the sample site is desired. The guide material is preferably biocompatible.

The guide is, optionally, coupled to the sampling site through the use of contact fluid, such as a fluorocarbon, a fluoropolymer, a fluorocompound, Fluorinert, FC-40, FC-70, or equivalent. Optionally, coupling fluids that index of refraction match are used.

Key (Attachment)

In its broadest sense, an attachment interfaces with a guide. Several attachments including a plug, photonic stimulator, a sample module, and a miniaturized source are previously described. The guide aids in reproducible positioning of the attachment in relation to the guide. The sample side curvature of the guide is independent of the guide's ability to interface with any of the attachments.

Preferably, there exists a commonality of the lock and key mechanism of the various guides and the various attachments for quick interchange and reproducible placement of the guide relative to the key. For example, the plug, photonic stimulator, and the miniaturized source, preferably, have the same key so that they all interface to the same guide lock. This allows the photonic stimulator or miniaturized source attachment to be rapidly and reproducibly aligned relative to the reference guide.

Plug

A plug is replaceably attached to a sample site. The plug is useful for a number of purposes, including hydration of the sample site and protection of the sample site.

One species of plugs are plugs attached to a guide with a key element that interfaces with the guide. For example, a hydration inducing plug is securely attached from the aperture of the guide to the mount at the contacting end. The outer surface of the hydration inducing plug is aligned with the mount's contact surface and is in direct contact with the sampling site. Preferably, the hydration inducing plug has an evenly flat member, the edge of which is securely attached to the mount of the guide. When the plug is coupled with the guide, the evenly flat member of the plug is in direct contact with the sample surface. When the optical probe is coupled into the aperture of the guide the surface of the plug acts to induce hydration of the sample surface and/or to protect the sample site. The hydration inducing plug is made of a material or materials, such as a plastic or a fluoropolymer, having properties that include at least one of being near-IR transmissive, hydrophobic, refractive index matching to skin, and insulating.

Alternatively, a plug is used without a guide and does not have an interface to a guide. In this case, the plug is replaceably attached to the sample site surface and acts to protect and/or hydrate the sample site

Adhesive

In its broadest sense, an adhesive acts to attach an apparatus to a sample site, or to attach an apparatus about the sample site, in a replaceable fashion. Examples of attached apparatus include a guide, a photostimulator, and a plug. Preferably, the adhesive is a double sided piece of tape. Alternative adhesives include tape, glue, hook and loop fasteners, straps, and bands, such as a watch band.

Alignment Tool

An alignment tool is used to align a guide onto an adhesive placed onto the arm. The pictured plug in FIG. 1 has an optional central tunnel. This tunnel is used in the initial placement of the guide. In this embodiment, a double sided adhesive strip is attached to the sampling site. The adhesive strip has an opening in it that is slightly larger than the optical probing element. After the adhesive is placed onto the sampling site, e.g. a subject's arm, the guide is attached to the plug and slid down a guiding rod to the adhesive so that the optical path is centered in the cutout on the adhesive. Essentially, the rod which is positioned through the plug and guide is used as a sighting mechanism.

Cleaner

Cleaning a sample site with soap and water is sometimes a problem in subsequent near-IR noninvasive glucose concentration determinations because the cleaner evaporates and leaves a near-IR absorbing residue that interferes with subsequent noninvasive glucose concentration determinations. Preferably, the sample site and/or the region about the sample site is cleaned. The sample site is cleaned for a number of reasons, including any of removing loose skin cells, filling air gaps in the skin with a fluid, to remove foreign objects from the surface of the skin, and to enhance pliability. The sample site and/or the region about the sample site is cleaned for a number of reasons, including any of enhancing the subsequent attachment of an adhesive and enhancing the pliability of the skin. A number of cleaners exist, including an alcohol, a weak acid solution, a skin softener, a glucose solvent, and a mixture of alcohol and a water based solvent. An example of a weak acid is boric acid. An example of a skin softener is mineral oil. An example of a glucose solvent is an aqueous cleaner or other highly polar solvent, such as water.

Preferably, cleaners come in individual use sealed packets. Alternatively, cleaners come in larger containers, such as a spray bottle. An applicator, such as a cotton swab is optionally included with the cleaner.

Contact Fluid

In its broadest sense herein, a contact fluid is a fluid that makes contact between the skin surface of a sample site and an attachment, such as an optical probe of a sampling module. Contact fluids serve purposes including any of displacing air pockets in the outer skin surface, increasing hydration of the sample site, providing thermal control, and refractive index matching of the skin surface and the attachment in proximate contact with the skin surface. In the case where refractive indices are matched, a contact fluid is also a coupling fluid.

T. Blank, G. Acosta, M. Mattu, S. Monfre, Fiber optic probe guide placement guide, U.S. Pat. No. 6,415,167 (Jul. 2, 2002) describe a contact fluid of one or more perfluoro compounds where a quantity of the contact fluid is placed at an interface of the optical probe and measurement site. M. Robinson, U.S. Pat. No. 6,152,876, supra and M. Rohrscheib, U.S. Pat. No. 6,240,306, supra describe a refractive index matching coupling fluids composed of chlorofluorocarbons. However, because the chlorine in a chlorofluorocarbon is associated with toxicity, it is preferable that the contact fluid be a fluorocarbon molecule, a fluorocarbon polymer, a fluorocompound, or a mixture of any of these despite a resulting mismatch in refractive index. Some specific examples of a fluorocarbon contact fluid are FC-40, FC-70, and FC-72 available from 3M. The index of refraction of FC-72, FC-40, and FC-70 is 1.251, 1.290, and 1.303, respectively. This is intermediate between skin with a refractive index of 1.44 and air with a refractive index of 1.0 and thus, according to Fresnel, increase the percentage of normally incident photons penetrating into the skin from air. However, the refractive indices of the fluorocarbons are not between skin with a refractive index of 1.44 and optical glasses with refractive indices greater than 1.5. Therefore, the fluorocarbons are not refractive index matching coupling fluids. Rather, a fluorocarbon fluid is a contact fluid.

Alternatively, the contact fluid serves the purpose of partially penetrating into the skin to provide better optical coupling to more internalized layers of skin. For example, the fluorinert wets the keratinocytes, displace air pockets in the stratum corneum, and generally levels the rough surface of skin.

A contact fluid is preferably near-IR inactive. An example of near-IR inactive is a fluid that does not absorb more than one percent in the region from 1100 to 1900 nm with a pathlength of less than 0.2 mm.

Alternatively, a contact fluid is used for thermal control. Surface skin temperature is dynamic. In one embodiment of the invention, a contact fluid is thermally controlled to a target temperature. The target temperature is from 85 to 98 degrees Fahrenheit and preferably 90±2 degrees Fahrenheit. The contact fluid, controlled to the target temperature, is then applied to the tissue sample site. This adjusts the outer surface of the skin temperature to a known temperature. Preferably, the target temperature is slightly less than body temperature. The tip of the sample probe is also controlled to this target temperature. Therefore, when the tip of the sample probe interfaces with the tissue sample site, a small temperature gradient exists between the tip of the sample probe and the tissue sample site. This small differences reduces thermal effects observed in the spectra and results in better precision and accuracy in glucose concentration determinations. Optionally, the reference is temperature controlled, such as to a target temperature.

Sterile Wipe

In its broadest sense, a sterile wipe is used to prepare a sample site surface and/or the area about the sample site. Preferably, the sterile wipe is in a container, such as a multi-sheet dispenser or in individually wrapped packages. Optionally, the container or individually wrapped packages are sealed or hermetically sealed. An example of a sterile wipe is an absorbent swab, such as a cotton swab or an artificial material.

Package

Preferably the kit elements are contained in one or more packages. The package contains the elements in single location and keeps the kit element clean.

Photonic Stimulator

The primary function of a photonic stimulator is to increase localized perfusion. The difference in glucose concentration in different body compartments and the importance of this difference to noninvasive glucose concentration calibration, maintenance, and prediction is presented in detail in U.S. patent application Ser. No. 10/377,916 (attorney docket no. SENS0004), which is herein incorporated in its entirety this by reference thereto.

Photo-stimulation is also referred to as photostimulation, photonic stimulation, or stimulation or excitation with light or photons. Photostimulation is herein used to refer to photons being absorbed by an absorber that subsequently releases an agent that results in increased perfusion. Photo-stimulation at or near the sample site is performed in a manner that enhances perfusion of the sample site primarily by enhancing or inducing perfusion of the sample site. Photostimulation is distinct from photonic heating. Photonic heating is optionally used in conjunction with photostimulation.

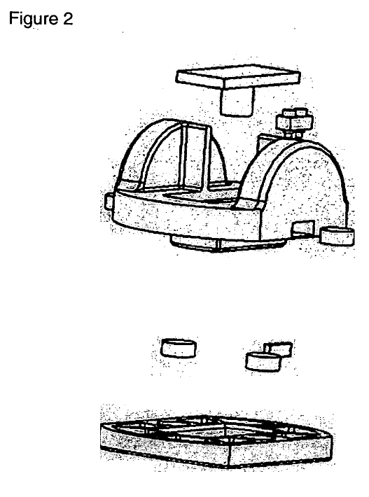

A photonic stimulator attachment is presented in FIG. 2. In this embodiment, the photonic stimulator 31 within an attachment apparatus 21 is coupled to a guide 10 with a 4.5 inch radius of curvature sample interface. Optionally, the guide has a flexible interface to the skin allowing the weight of an attachment to the guide to be displaced over a larger area. Photo-stimulation at or near at least one sample site is used to enhance perfusion of the sample site leading to reduced errors associated with sampling. Increased perfusion of the sample site leads to increased volume percentages of the target analyte and/or allows the blood or tissue constituent concentrations to more accurately and/or precisely track corresponding sample constituents in more well perfused body compartments or sites, such as arteries, veins, or fingertips. In one embodiment, analysis of the photo-stimulated site is used in conjunction with glucose concentration analyzers to determine the glucose analyte concentration with greater ease, accuracy, or precision and allows determination of the analyte concentration of another non-sampled body part or compartment. This technology is described in U.S. patent application Ser. No. 10/841,200 (attorney docket no. SENS0034) and is herein incorporated in its entirety by this reference thereto.

In the preferred embodiment, the photonic stimulator is incorporated into an attachment that fits into a guide, as shown in FIG. 2. In an alternative embodiment, the LED's are automatically turned on when the attachment is placed into the guide. In this case, the copper insert in the guide completes a contact with the metal pins of the attachment. A battery is placed into the photonic stimulator guide. Optionally, the attachment is configured with a button or switch to manually power on/off the source. Optionally, the power to the LED's runs from a base module to the attachment.

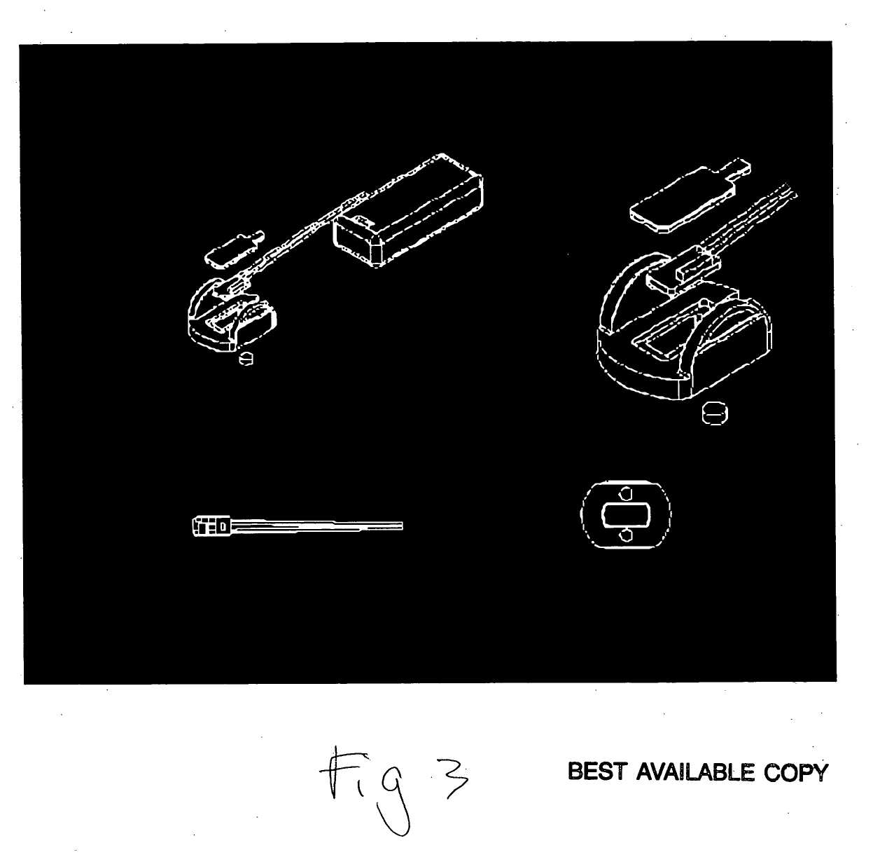

An additional embodiment of a photonic stimulator placed into a guide is provided here. A photonic stimulator attachment is presented in FIG. 3 coupled to a guide. In the embodiment pictured, a guide 10 is coupled to a plug 11. The plug contains three light emitting diodes (LEDs) 31 along with a circuit board. Alternatively, one or more LED's are provided. Power is supplied via an auxiliary battery or power pack. Alternatively, the power supply is integrated into the plug. In this example, magnets 12 are used to facilitate reproducible alignment between the guide and the plug and hence between the plug containing the LEDs and the sample site.

The photonic stimulator attachment results in many of the advantages or properties of a plug. The photonic stimulator attachment is optionally used as a plug to accomplish at least one of hydration of the sampling site by occlusion, protection of the sampling site from physical perturbation, protection of the sampling site from contamination, alignment of the guide, and allowing an aesthetic appearance, such as a watch, ring, or graphical symbol.

Although the invention is described herein with reference to the preferred embodiments, one skilled in the art will readily appreciate that other applications may be substituted for those set forth herein without departing from the spirit and scope of the present invention. Accordingly, the invention should only be limited by the claims included below.

1. A sample site preparation kit for use in conjunction with a near-infrared, noninvasive analyte concentration analyzer, comprising:
a guide for coupling said analyzer to a targeted and controlled sample site; and
a plug for protecting said sample site.

2. A sample site preparation kit for use in conjunction with a near-infrared, noninvasive glucose concentration analyzer, comprising:
a guide, having an attachment surface, for aligning said noninvasive glucose analyzer to a sample site; and
an adhesive for attaching said attachment surface of said guide at or near said sample site.

3. The kit of claim 2, wherein said adhesive comprises at least one of tape and glue.

4. The kit of claim 3, wherein said tape comprises double sided tape.

5. A sample site preparation kit for use in conjunction with a near-infrared, noninvasive glucose concentration analyzer, comprising:
a guide, having a first and second edge, for aligning an interface of said noninvasive analyzer to a targeted and controlled sample site; and
means for attaching said guide to said sample site.

6. The kit of claim 5, wherein said attaching means comprise at least one of:
a strap, having a first and second part, wherein said first part of said strap attaches to said first edge, said strap wraps around a body part, and said second part attaches to said second edge;
glue;
double sided tape; and
loop and hook fastener.

7. A sample site preparation kit for use in conjunction with a near-infrared, noninvasive glucose concentration analyzer, comprising:
a guide for aligning said analyzer to a targeted sample site; and
a cleaner for cleaning at least one of:
a sample site;
a region about a sample site; and
said region and said sample site.

8. The kit of claim 7, wherein said cleaner comprises any of:
alcohol;
a sterile wipe;
a weak acid solution;
a skin softener;
a glucose solvent; and
a mixture of alcohol and a water based solvent.

9. The kit of claim 7, further comprising:
a sealed packet for containing said cleaner.

10. A kit for use in conjunction with a near-infrared, noninvasive glucose concentration analyzer, comprising:
a guide for aligning said analyzer to a targeted sample site; and
a contact fluid for optically coupling said analyzer to said sample site.

11. The kit of claim 10, wherein said fluid comprises at least one of:
a fluoropolymer;
a fluorocarbon;
a chlorofluorcarbon; and
a mixture of said fluorocarbon and a diluent.

12. The kit of claim 10, wherein said fluid does not absorb more than one percent in the near-infrared region from 1100 to 1900 nm with a pathlength of less than 0.2 mm.

13. A kit for use in conjunction with a near-infrared, noninvasive glucose concentration analyzer, comprising:
a guide for aligning said analyzer to a targeted and controlled sample site; and
a photostimulator for increasing perfusion of said sample site.

14. A sample site preparation kit for use in conjunction with a near-infrared, noninvasive glucose concentration analyzer, comprising:
a plug for expediting hydration of a sample site; and
an adhesive for attaching said plug to said sample site.

15. A sample site preparation kit for use in conjunction with a near-infrared, noninvasive glucose concentration analyzer, comprising:
a plug for expediting hydration of a sample site; and
a cleaner comprising at least one of:
alcohol;
a sterile wipe;
a weak acid solution;
a skin softener;
a glucose solvent; and
a mixture of alcohol and a water based solvent.

16. A sample site preparation kit for use in conjunction with a near-infrared, noninvasive glucose concentration analyzer, comprising:
a plug for expediting hydration of a sample site; and
a fluid in, a container, for optically coupling said analyzer to said sample site.

17. The kit of claim 16, wherein said fluid comprises at least one of:
a fluoropolymer;
a fluorocarbon;
a chlorofluorcarbon; and
a mixture of said fluorocarbon and a diluent.

18. A kit for use in conjunction with a near-infrared, noninvasive glucose concentration analyzer, comprising a package containing at least two of:
a guide, having an attachment surface for aligning an interface of said noninvasive analyzer to a targeted and controlled sample site;
means for attaching said guide to a region about said sample site;
a plug for expediting hydration of said sample site;
a cleaning fluid for cleaning at least one of a sample site and a region about said sample site;
a razor for shaving hair from said sample site;
a contact fluid, in a container for interfacing said analyzer to said sample site;
a photostimulator for dilating capillaries of said sample site; and
an alignment tool.

19. The kit of claim 18, wherein said container further comprises a hermetic seal.

20. The kit of claim 18, wherein said contact fluid comprises a coupling fluid.

21. The kit of claim 18, wherein said adhesive comprises at least one of:
tape having a first side and a second side; and
a glue.

22. The kit of claim 21, wherein said first side of said tape affixes to said attachment surface of said guide.

23. The kit of claim 21, further comprising a peel off layer attached to said second side of said tape.

24. The kit of claim 18, wherein said means for attaching comprises at least one of:
a strap having a first and second part, wherein said first part of said strap attaches to said first edge, said strap wraps around a body part, and said second part attaches to said second edge,
glue;
double sided tape; and
a loop and hook fastener.

25. The kit of claim 24, wherein said cleaning fluid enhances attachment of said double sided tape to said region about said sample site.

26. A sample probe preparation kit for use in conjunction with a near-infrared, noninvasive glucose concentration analyzer with an optical interface tip, comprising:
a cleaning solution; and
a wipe;
wherein said cleaning solution and said wipe clean said optical interface tip of said analyzer.

27. The kit of claim 26, wherein said cleaning solution comprises at least one of:
an alcohol based solvent;
an aqueous based solvent; and
a solvent containing at least alcohol and water.

28. A sample site preparation kit for use in conjunction with a near-infrared, noninvasive analyte concentration analyzer, comprising:
a guide having a top surface and a contact surface, wherein at least part of said contact surface of said guide comprises a flexible member; and
a plug for contacting said top surface of said guide and for hydrating said sample site.

29. A sample site preparation kit for use in conjunction with a near-infrared, noninvasive analyte concentration analyzer, comprising:
a guide having a top surface and a contact surface, wherein at least part of said contact surface of said guide comprises a flexible element; and
an attachment for coupling to said guide.

30. The kit of claim 29, wherein said attachment comprises at least one of:
a plug, a sample module, and a photostimulator.

31. A kit for use in conjunction with a near-infrared, noninvasive glucose concentration analyzer, comprising a guide and at least two of:
means for attaching said guide to said sample site;
a plug for expediting hydration of said sample site;
a cleaning fluid for cleaning comprising at least one of a sample site and a region about said sample site;
a razor for shaving hair from said sample site;
a contact fluid, in a container, for interfacing said analyzer to said sample site;
a photostimulator for dilating capillaries of said sample site; and
an alignment tool.

32. A sample site preparation kit for use in conjunction with a near-infrared, noninvasive glucose concentration analyzer, comprising:
a cleaning fluid, in a container, for cleaning at least one of a sample site and a region about a sample site; and
a contact fluid, in a container, for interfacing said analyzer to said sample site.

33. The kit of claim 32, wherein said cleaning fluid comprises at least one of:
alcohol;
a sterile wipe;
a weak acid solution;
a skin softener;
a glucose solvent; and
a mixture of alcohol and a water based cleaner.

34. A sample site preparation kit for use in conjunction with a near-infrared, noninvasive analyte concentration analyzer, comprising:
a guide having a contact surface and an aperture wall; and
a plug for replaceably contacting said aperture wall of said guide and for hydrating said sample site.

35. The kit of claim 34, wherein at least part of said contact surface of said guide comprises a flexible member.

36. The kit of claim 34, wherein at least part of said guide comprises a flexible material.

37. A disposable kit for use in conjunction with a near-infrared, noninvasive glucose analyzer, comprising at least one of:
a guide for aligning an interface of said noninvasive analyzer to a targeted and controlled sample site;
means for attaching an attachment to said sample site;
a plug for expediting hydration of said sample site;
a cleaning fluid for cleaning at least one of a sample site and a region about said sample site;
a contact fluid, in a container, for interfacing said analyzer to said sample site;
a photostimulator for dilating capillaries of said sample site; and
an alignment tool.
